(19) United States
(12) Patent Application Publication
(10) Pub. No.: US 2016/0058291 A1
(43) Pub. Date: Mar. 3, 2016

(54) Photoacoustic Imager

(71) Applicant: XTrillion, Inc., Tokyo (JP)

(72) Inventor: Hitoshi NAKATSUKA, Tokyo (JP)

(21) Appl. No.: 14/790,290

(22) Filed: Jul. 2, 2015

(30) Foreign Application Priority Data
Aug. 27, 2014 (JP) 2014-172734

(51) Int. Cl. A61B 5/00; H05B 33/08

(52) U.S. Cl. CPC A61B 5/0095; H05B 33/0887; A61B 5/725; A61B 5/742

(57) ABSTRACT

In this photoacoustic imager, a light source driving portion is configured to stop power supply to a plurality of light-emitting semiconductor elements on the basis of that a forward voltage detection portion has detected forward voltage values of the plurality of light-emitting semiconductor elements corresponding to short circuiting of the light-emitting semiconductor elements.

### BACKGROUND OF THE INVENTION

1. Field of the Invention

The present invention relates to a photoacoustic imager, and more particularly, it relates to a photoacoustic imager including light-emitting semiconductor elements capable of emitting light.

2. Description of the Background Art

A photoacoustic imager including light-emitting semiconductor elements capable of emitting light is known in general, as disclosed in Japanese Patent Laying-Open No. 2010-103391, for example.

The aforementioned Japanese Patent Laying-Open No. 2010-103391 discloses an LED lighting device including a plurality of serially connected LEDs and an overheat protection resistor (thermistor), connected to the plurality of LEDs, whose resistance value varies with temperature increase/decrease. This LED lighting device is so configured that load current flowing in the plurality of LEDs increases when any one of the plurality of LEDs is short-circuited thereby overheating the plurality of LEDs and increasing the resistance value of the overheat protection resistor. The load current flowing in the plurality of LEDs decreases due to the increase in the resistance value of the overheat protection resistor, whereby the LEDs are prevented from thermal breakage resulting from overheating.

Also when any one of the plurality of LEDs is short circuited, however, the LED lighting device according to the aforementioned Japanese Patent Laying-Open No. 2010-103391 continuously emits light in the state where the resistance value of the overheat protection resistor increases (the load current decreases). Therefore, the LED lighting device disadvantageously continues the emission of light in a state where the quantities of light from the plurality of LEDs (light-emitting semiconductor elements) are insufficient.

### SUMMARY OF THE INVENTION

The present invention has been proposed in order to solve the aforementioned problem, and an object of the present invention is to provide a photoacoustic imager capable of preventing continuation of light emission in a state where the quantities of light from light-emitting semiconductor elements are insufficient.

In order to attain the aforementioned object, a photoacoustic imager according to an aspect of the present invention includes a detection/generation portion capable of detecting an acoustic wave generated due to absorption of light by a detection object in a specimen and generating an ultrasonic wave to be applied to the specimen, a plurality of light-emitting semiconductor elements provided in the vicinity of the detection/generation portion and capable of applying light to the specimen, a light source driving portion supplying power for making the light-emitting semiconductor elements generate light to the light-emitting semiconductor elements and a forward voltage detection portion capable of detecting forward voltage values of the light-emitting semiconductor elements, while the light source driving portion is configured to stop power supply to the light-emitting semiconductor elements on the basis of that the forward voltage detection portion has detected forward voltage values of the plurality of light-emitting semiconductor elements corresponding to short circuiting of the light-emitting semiconductor elements.

In the photoacoustic imager according to the aspect of the present invention, as hereinabove described, the light source driving portion is configured to stop power supply to the light-emitting semiconductor elements on the basis of that the forward voltage detection portion has detected the forward voltage values of the plurality of light-emitting semiconductor elements corresponding to short circuiting of the light-emitting semiconductor elements. When any one of the plurality of light-emitting semiconductor elements is short-circuited, the total forward voltage value of the plurality of light-emitting semiconductor elements is reduced in response thereto. Therefore, the light source driving portion stops power supply to the light-emitting semiconductor elements on the basis of that the forward voltage detection portion has detected the forward voltage values of the plurality of light-emitting semiconductor elements corresponding to short circuiting of the light-emitting semiconductor elements as hereinabove described so that the light-emitting semiconductor elements are prevented from emitting light in a state where any one of the plurality of light-emitting semiconductor elements is short-circuited, whereby the photoacoustic imager can be prevented from continuously emitting light in a state where the quantities of light from the light-emitting semiconductor elements are insufficient.

In the photoacoustic imager according to the aforementioned aspect, the light source driving portion is preferably configured to stop power supply to the light-emitting semiconductor elements in a case where the forward voltage values of the plurality of light-emitting semiconductor elements detected by the forward voltage detection portion are not more than a prescribed threshold. Even if one or more of the light-emitting semiconductor elements are short-circuited, the quantities of light may not be insufficient (within tolerance). In this case, the light source driving portion is configured to stop power supply to the light-emitting semiconductor elements in the case where the forward voltage values of the plurality of light-emitting semiconductor elements detected by the forward voltage detection portion are not more than the prescribed threshold as described above, whereby the same can properly stop power supply to the light-emitting semiconductor elements when the quantities of light are insufficient (out of tolerance).

In the photoacoustic imager according to the aforementioned aspect, the prescribed threshold is preferably set to a forward voltage value in a case where at least two light-emitting semiconductor elements are short-circuited. According to this structure, the light source driving portion can be prevented from stopping power supply to the light-emitting semiconductor elements when the number of short-circuited light-emitting semiconductor elements corresponds to that causing no insufficiency in the quantities of light.

In the photoacoustic imager according to the aforementioned aspect, the light source driving portion is preferably configured to stop power supply to the light-emitting semiconductor elements on the basis of that the forward voltage detection portion has detected the forward voltage values of the plurality of light-emitting semiconductor elements corresponding to short circuiting of the light-emitting semiconductor elements at least in an OFF-period when the light-emitting semiconductor elements emit no light. According to this structure, the forward voltage detection portion can easily detect the forward voltage values dissimilarly to a case of detecting the forward voltage values of the plurality of light-emitting semiconductor elements only in a relatively short ON-period when the light-emitting semiconductor elements emit light.

In this case, the light source driving portion is preferably configured to stop power supply to the light-emitting semiconductor elements in both of a case where the forward voltage values of the plurality of light-emitting semiconductor elements detected by the forward voltage detection portion are not more than a first threshold in an ON-period when the light-emitting semiconductor elements emit light and a case where the forward voltage values of the plurality of light-emitting semiconductor elements detected by the forward voltage detection portion are not more than a second threshold in an OFF-period when the light-emitting semiconductor elements emit no light. According to this structure, the light source driving portion stops power supply to the light-emitting semiconductor elements in both of ON- and OFF-periods in a case where the light-emitting semiconductor elements are short-circuited, whereby the photoacoustic imager can be more prevented from continuously emitting light in a state where the quantities of light from the light-emitting semiconductor elements are insufficient.

In the aforementioned photoacoustic imager in which the light source driving portion stops power supply on the basis of that the forward voltage detection portion has detected the forward voltage values at least in an OFF-period, the light source driving portion is preferably configured to stop power supply to the light-emitting semiconductor elements in a case where the forward voltage values of the plurality of light-emitting semiconductor elements detected by the forward voltage detection portion are not more than a second threshold only in an OFF-period when the light-emitting semiconductor elements emit no light. When the forward voltage detection portion detects the forward voltage values of the plurality of light-emitting semiconductor elements in a relatively short ON-period when the light-emitting semiconductor elements emit light, the photoacoustic imager may separately require a circuit (a sample hold circuit, for example) holding the forward voltages value until the forward voltage detection portion performs an operation of detecting the forward voltage values in practice. Therefore, the light source driving portion is configured to stop power supply to the light-emitting semiconductor elements on the basis of the forward voltage values of the plurality of light-emitting semiconductor elements detected by the forward voltage detection portion only in an OFF-period when the light-emitting semiconductor elements emit no light as described above so that the forward voltage detection portion detects the forward voltage values of the plurality of light-emitting semiconductor elements in a relatively long OFF-period, whereby the forward voltage detection portion can easily detect the forward voltage values with no requirement for a circuit for holding the forward voltage values.

The photoacoustic imager according to the aforementioned aspect preferably further includes a low-pass filter provided between the light-emitting semiconductor elements and the forward voltage detection portion, and the light source driving portion is preferably configured to stop power supply to the light-emitting semiconductor elements on the basis of that the forward voltage detection portion has detected the forward voltage values of the plurality of light-emitting semiconductor elements corresponding to short circuiting of the light-emitting semiconductor elements through the low-pass filter. According to this structure, the (relatively large) forward voltage values in an ON-period when the light-emitting semiconductor elements emit light and the (relatively small) forward voltage values in an OFF-period when the light-emitting semiconductor elements emit no light are rendered substantially constant through the low-pass filter, whereby the forward voltage detection portion can easily detect the forward voltage values regardless of an ON- or OFF-period.

In this case, the light source driving portion is preferably configured to stop power supply to the light-emitting semiconductor elements in a case where the forward voltage values of the plurality of light-emitting semiconductor elements detected by the forward voltage detection portion through the low-pass filter are not more than a common third threshold in an ON-period when the light-emitting semiconductor elements emit light and in an OFF-period when the light-emitting semiconductor elements emit no light. The forward voltage detection portion detects the forward voltage values through the low-pass filter for rendering the forward voltage values in an ON-period and those in an OFF-period substantially constant, whereby the light source driving portion can properly stop power supply to the light-emitting semiconductor elements by employing the third threshold.

The photoacoustic imager according to the aforementioned aspect preferably further includes a display portion displaying states of the light-emitting semiconductor elements on the basis of that the forward voltage detection portion has detected the forward voltage values of the plurality of light-emitting semiconductor elements corresponding to short circuiting of the light-emitting semiconductor elements. According to this structure, the photoacoustic imager can easily make the user visually recognize the states of the light-emitting semiconductor elements (the number of short-circuited light-emitting semiconductor elements based on the forward voltage values, for example). Consequently, the photoacoustic imager can prompt the user to exchange the light-emitting semiconductor elements.

In this case, the photoacoustic imager preferably further includes a control portion calculating the number of short-circuited light-emitting semiconductor elements on the basis of the forward voltage values of the plurality of light-emitting semiconductor elements detected by the forward voltage detection portion and a forward voltage value per light-emitting semiconductor element, and the display portion is preferably configured to display the number of the short-circuited light-emitting semiconductor elements calculated by the control portion. According to this structure, the photoacoustic imager can make the user recognize that an exchange period for the light-emitting semiconductor elements is approaching.

The aforementioned photoacoustic imager including the display portion preferably further includes a storage portion storing states of the light-emitting semiconductor elements and a control portion calculating an exchange period for the light-emitting semiconductor elements on the basis of the states of the light-emitting semiconductor elements stored in the storage portion, and the display portion is preferably configured to display the exchange period for the light-emitting semiconductor elements calculated by the control portion. According to this structure, the photoacoustic imager can make the user prepare new light-emitting semiconductor elements for exchange.

In the aforementioned photoacoustic imager including the display portion, the display portion is preferably configured to display the states of the light-emitting semiconductor elements when the photoacoustic imager is driven or power is applied to the photoacoustic imager. According to this structure, the photoacoustic imager can easily make the user recognize the states of the light-emitting semiconductor elements.

The photoacoustic imager according to the aforementioned aspect preferably further includes a switch portion having a first end connected to the light-emitting semiconductor elements and a grounded second end, and the switch portion preferably consists of a field-effect transistor. Small current (leakage current) flows in the field-effect transistor also in an OFF state, and hence the forward voltage values have constant magnitudes also in an OFF-period when the light-emitting semiconductor elements emit no light. In other words, the magnitudes of the forward voltage values vary with a case where the light-emitting semiconductor elements are short-circuited and a case where the same are not short-circuited also in an OFF-period when the light-emitting semiconductor elements emit no light. When the switch portion is constituted of the field-effect transistor, therefore, short circuiting of the light-emitting semiconductor elements can be detected also in an OFF-period when the light-emitting semiconductor elements emit no light.

In the photoacoustic imager according to the aforementioned aspect, the forward voltage detection portion is preferably provided between the light source driving portion and the light-emitting semiconductor elements. According to this structure, the forward voltage detection portion can easily acquire the forward voltage values of the light-emitting semiconductor elements on the basis of the difference between the voltage of the light-source driving portion and those of the light-emitting semiconductor elements.

In the photoacoustic imager according to the aforementioned aspect, the forward voltage detection portion is preferably configured to detect the sum of the forward voltage values of the plurality of light-emitting semiconductor elements. The forward voltage value per light-emitting semiconductor element is previously knowable, and hence the control portion can easily calculate the number of short-circuited light-emitting semiconductor elements from the sum of the forward voltage values of the plurality of light-emitting semiconductor elements. In other words, the control portion can calculate the number of short-circuited light-emitting semiconductor elements without detecting the individual forward voltage values of the plurality of light-emitting semiconductor elements.

In the photoacoustic imager according to the aforementioned aspect, the forward voltage detection portion is preferably configured to acquire voltage values on the basis of a sampling trigger signal synchronized with a light trigger signal controlling ON- and OFF-states of the light-emitting semiconductor elements. According to this structure, the forward voltage detection portion can acquire the voltage values in synchronization with ON- and OFF-states of the light-emitting semiconductor elements.

In the photoacoustic imager according to the aforementioned aspect, the light-emitting semiconductor elements are preferably constituted of light-emitting diode elements. According to this structure, the range of light application is relatively hard to change even if misregistration is caused, since the light-emitting diode elements are lower in directivity as compared with light-emitting semiconductor elements emitting laser beams. Thus, the photoacoustic imager requires neither precise alignment (registration) of optical members nor an optical bench or a strong housing for preventing characteristic fluctuation resulting from vibration of optical systems, dissimilarly to a case of employing light-emitting semiconductor elements emitting laser beams. Consequently, the photoacoustic imager can be prevented from size increase and complication in structure due to the nonrequirement for precise alignment of optical members and for an optical bench or a strong housing.

In the photoacoustic imager according to the aforementioned aspect, the light-emitting semiconductor elements are preferably constituted of semiconductor laser elements. According to this structure, the semiconductor laser elements can apply laser beams relatively higher in directivity as compared with the light-emitting diode elements to the specimen, whereby most part of light from the semiconductor laser elements can be reliably applied to the specimen.

In the photoacoustic imager according to the aforementioned aspect, the light-emitting semiconductor elements are preferably constituted of organic light-emitting diode elements. According to this structure, the light source portion provided with the light-emitting semiconductor elements can be easily miniaturized by employing the organic light-emitting diode elements easily reducible in thickness.

According to the present invention, as hereinabove described, the photoacoustic imager can be prevented from continuation of light emission in a state where the quantities of light from the light-emitting semiconductor elements are insufficient.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a block diagram showing the overall structure of a photoacoustic imager according to a first (second) embodiment of the present invention;

FIG. 2 is a block diagram showing the structure of part of the photoacoustic imager according to the first (second) embodiment of the present invention;

FIG. 3 is a timing chart for illustrating operations of the photoacoustic imager according to the first embodiment of the present invention;

FIG. 4 is a diagram for illustrating characteristics of a light-emitting diode element (relation between a forward voltage value and a current value) according to the first embodiment of the present invention;

FIG. 5 is a block diagram showing the structure of part of a photoacoustic imager according to a third embodiment of the present invention;

FIG. 6 is a timing chart for illustrating operations of the photoacoustic imager according to the third embodiment of the present invention;

FIG. 7 is a block diagram showing the overall structure of a photoacoustic imager according to a fourth embodiment of the present invention;

FIG. 8 is a diagram showing information of light-emitting diode elements displayed on an image display portion of the photoacoustic imager according to the fourth embodiment of the present invention; and

FIG. 9 is a block diagram showing the structure of part of a photoacoustic imager according to a first modification (second modification) of the first embodiment of the present invention.

### DESCRIPTION OF THE PREFERRED EMBODIMENTS

Embodiments of the present invention are now described with reference to the drawings.

### First Embodiment

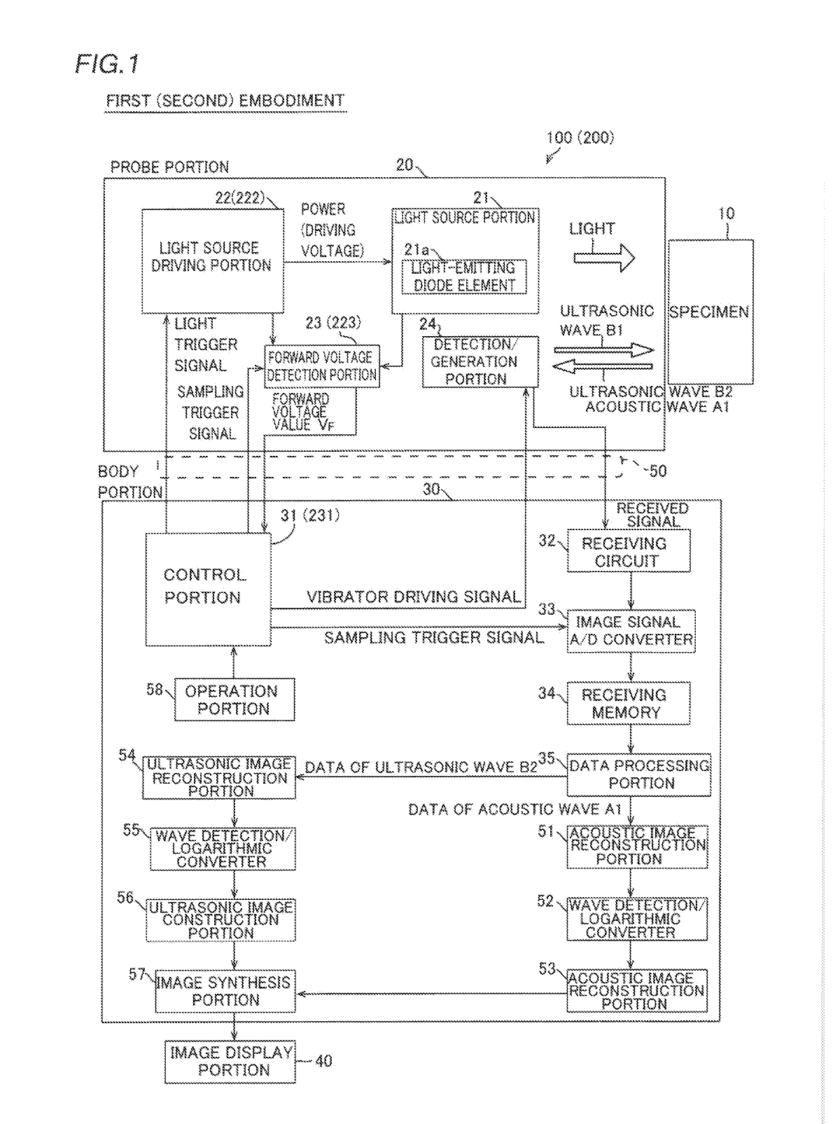

The structure of a photoacoustic imager 100 according to a first embodiment of the present invention is described with reference to FIGS. 1 to 4.

The photoacoustic imager 100 according to the first embodiment of the present invention is provided with a probe portion 20, as shown in FIG. 1. The probe portion 20 is linearly formed, and configured to be capable of emission of light and transmission of ultrasonic waves B1 and B2 described later when the same is brought into contact with a specimen 10 (surface of a human body) by an operator. Further, the probe portion 20 is configured to detect an acoustic wave A1 and the ultrasonic wave B2 from the specimen 10 and to transmit the same as received signals to a body portion 30 described below through a cable 50 of about 2 m.

The photoacoustic imager 100 is also provided with the body portion 30. The body portion 30 is configured to process and image the received signals detected by the probe portion 20.

The photoacoustic imager 100 is further provided with an image display portion 40. The image display portion 40 is configured to be capable of acquiring and displaying images processed by the body portion 30.

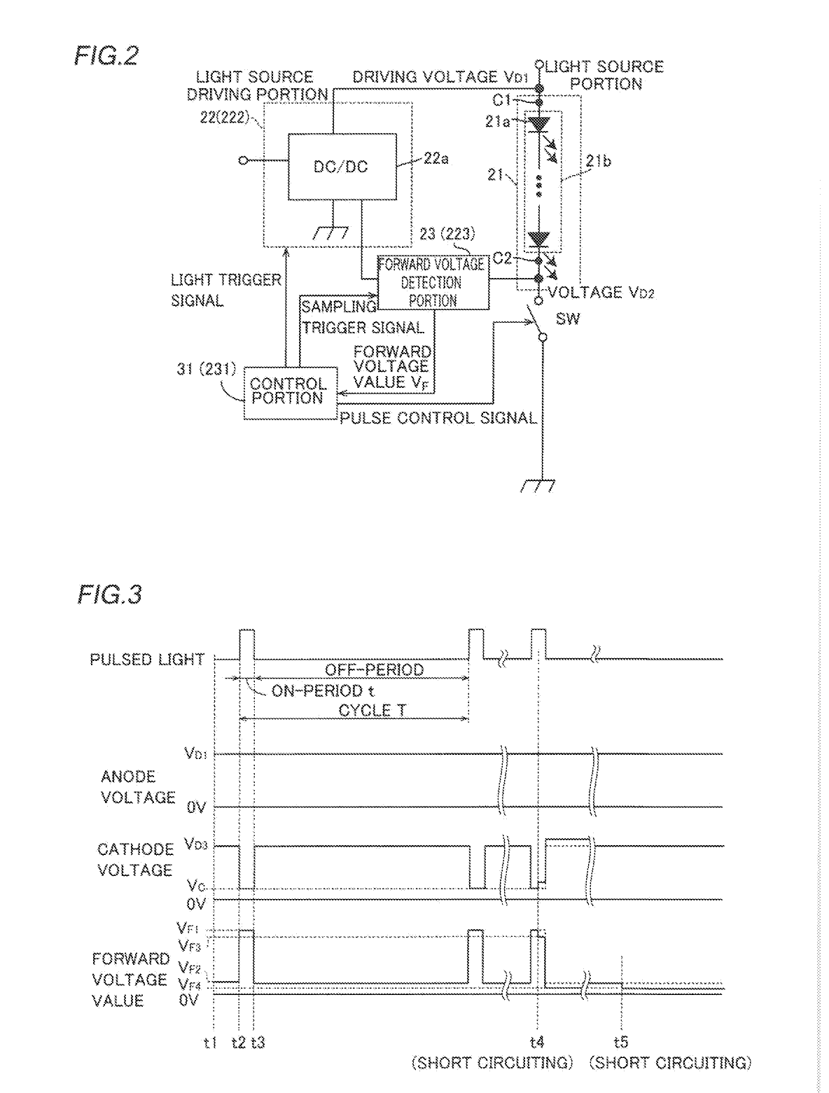

As shown in FIGS. 1 and 2, the probe portion 20 is provided with a light source portion 21. The light source portion 21 is provided with a plurality of light-emitting diode elements 21a capable of emitting pulsed light having a wavelength (about 850 nm, for example) in the infrared region easily infiltrating the specimen 10. The light-emitting diode elements 21a are examples of the “light-emitting semiconductor elements” in the present invention.

The probe portion 20 is also provided with a light source driving portion 22, a shown in FIG. 2. The light source driving portion 22 is provided with a DC-DC converter 22a. The light source driving portion 22 (the DC-DC converter 22a) is configured to supply power to the light source portion 21 on the basis of a light trigger signal received from a control portion 31 described later. More specifically, the light source driving portion 22 is configured to apply driving voltage V_{D1} (about 250 V, for example) to anode sides of the light-emitting diode elements 21a of the light source portion 21.

The plurality of (e.g. 36) light-emitting diode elements 21a are serially connected with each other, thereby constituting a plurality of light-emitting diode element groups 21b. The plurality of light-emitting diode element groups 21b are parallelly connected with each other. The DC-DC converter 22a applies voltage to the anode sides (points C1) of the plurality of light-emitting diode elements 21a. Cathode sides (points C2) of the plurality of light-emitting diode elements 21a are connected to a first end of a switch portion SW. A second end of the switch portion SW is grounded. The switch portion SW is constituted of an FET (field-effect transistor), for example. The switch portion SW is configured to be capable of switching grounded and nongrounded states of the light-emitting diode elements 21a on the basis of a pulse control signal received from the control portion 31.

Thus, potential difference is caused between the anode sides and the cathode sides of the plurality of light-emitting diode elements 21a when the switch portion SW is in an ON-state (conducting state), whereby current flows in the plurality of light-emitting diode elements 21a, which in turn emit light. In other words, the light source driving portion 22 (the DC-DC converter 22a) supplies power to the light-emitting diode element groups 21b in this case. When the switch portion SW is in an OFF-state (disconnected state), no current flows in the plurality of light-emitting diode elements 21a. In other words, the light source driving portion 22 (the DC-DC converter 22a) stops power supply to the light-emitting diode element groups 21b in this case.

The photoacoustic imager 100 is configured to on-off control the switch portion SW with the pulse control signal transmitted from the control portion 31 described later, so that the potential difference between the points C1 and C2 of the light-emitting diode element groups 21b has a driving pulse (driving voltage) in the form of a rectangular wave. The pulse control signal is set to such a value that an ON-period t (see FIG. 3) is about 100 nm, for example, a repetition frequency (1/cycle T, see FIG. 3) is 1 kHz or the like, heat generation in the light-emitting diode elements 21a is small and the light-emitting diode elements 21a can efficiently emit light.

According to the first embodiment, the light source driving portion 22 is configured to stop power supply to the light-emitting diode elements 21a on the basis of that a forward voltage detection portion 23 described below has detected forward voltage values V_{F} of the plurality of light-emitting diode elements 21a corresponding to short circuiting of the light-emitting diode elements 21a. Details of the forward voltage values V_{F} and operations of the light source driving portion 22 are described later.

The probe portion 20 is further provided with the forward voltage detection portion 23. The forward voltage detection portion 23 is connected to the light source driving portion 22 (the DC-DC converter 22a) and the cathode sides of the light-emitting diode elements 21a of the light source portion 21, and configured to be capable of acquiring a voltage value obtained by differentiating the voltage (the driving voltage V_{D1}) on the side of the light source driving portion 22 and voltages (voltages V_{D2}) on the cathode sides of the light-emitting diode elements 21a. In other words, the forward voltage detection portion 23 is configured to be capable of acquiring the sum (forward voltage value V_{F}) of the forward voltage values of the plurality of light-emitting diode elements 21a (the light-emitting diode element groups 21b) included in the light source portion 21. Further, the forward voltage detection portion 23 is configured to acquire voltage values on the basis of a sampling trigger signal synchronized with the light trigger signal received from the control portion 31. In the following description, the “the sum of the forward voltage values of the plurality of light-emitting diode elements 21a (the light-emitting diode element groups 21b) included in the light source portion 21” is simply referred to as “the forward voltage value V_{F}”. The forward voltage detection portion 23 is further configured to transmit the acquired forward voltage value V_{F} to the control portion 31.

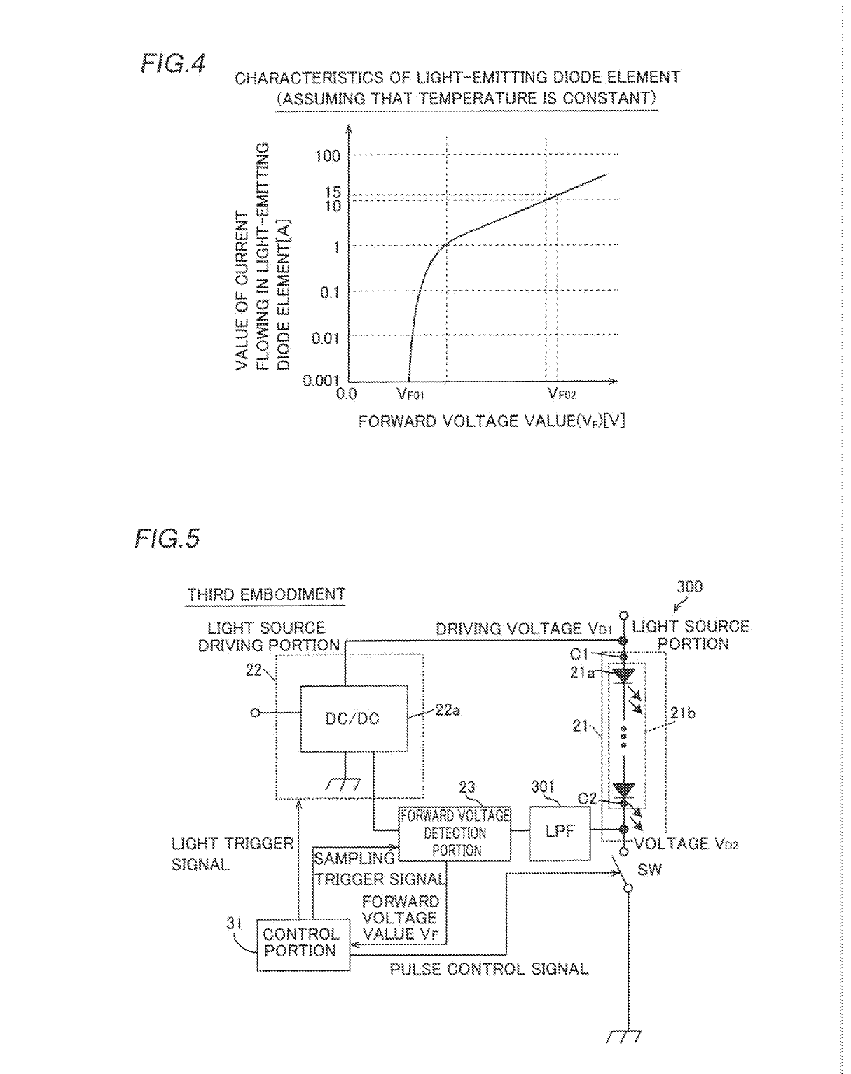

As shown in FIG. 4, the value of current flowing in the light-emitting diode elements 21a and the forward voltage value V_{F} are correlated with each other, assuming that the temperature (junction temperature) is constant.

As shown in FIG. 1, the light-emitting diode elements 21a are configured to apply light toward the specimen 10. A detection object (hemoglobin or the like, for example) in the specimen 10 absorbs the pulsed light applied from the probe portion 20 to the specimen 10. The detection object (the specimen) expands and contracts (returns to the original size from the expanded state) in response to the intensity of application (the quantity of absorption) of the pulsed light, thereby generating the acoustic wave A1. Throughout the specification, an ultrasonic wave generated due to absorption of light by the detection object in the specimen 10 is referred to as the “acoustic wave A1”, while an ultrasonic wave generated by the detection/generation portion 24 and reflected by the specimen 10 is referred to as the “ultrasonic wave B2” described later.

The probe portion 20 is further provided with the detection/generation portion 24. The detection/generation portion 24 includes an ultrasonic vibrator (not shown), and is configured to be capable of detecting the acoustic wave A1 from the specimen 10 and generating the ultrasonic wave B1 to be applied to the specimen 10. The ultrasonic vibrator is constituted of a piezoelectric element (lead zirconate titanate (PZT), for example) or the like. Further, the detection/generation portion 24 is configured to vibrate and generate voltage (received signal) when acquiring the aforementioned acoustic wave A1. The detection/generation portion 24 is also configured to transmit the acquired received signal to a receiving circuit 32 described later.

The detection/generation portion 24 (the ultrasonic vibrator) is further configured to be capable of generating the ultrasonic wave B1 by vibrating at a frequency responsive to a vibrator driving signal received from the control portion 31.

Further, the detection/generation portion 24 is configured to transmit a received signal to the receiving circuit 32 also in a case where the same vibrates due to the ultrasonic wave B2, similarly to the case where the same vibrates due to the ultrasonic wave B1.

The body portion 30 is provided with the control portion 31. The control portion 31 includes a CPU (central processing unit) or the like, and is configured to control the overall photoacoustic imager 100 by transmitting control signals to respective portions. For example, the control portion 31 is configured to transmit the light trigger signal to the light source driving portion 22. Thus, the light source driving portion 22 supplies power based on the light trigger signal to the light source portion 21, so that the light source portion 21 emits the pulsed light.

The body portion 30 is also provided with the receiving circuit 32. The receiving circuit 32 includes a coupling capacitor or the like, and is configured to acquire the received signal (alternating component) from the detection/generation portion 24. Further, the receiving circuit 32 is configured to transmit the acquired received signal to an image signal A/D converter 33.

The body portion 30 is also provided with the image signal A/D converter 33. The image signal A/D converter 33 is configured to convert the received signal (analog signal) acquired from the receiving circuit 32 to a digital signal correspondingly to the sampling trigger signal acquired from the control portion 31. Further, the image signal A/D converter 33 is connected with a receiving memory 34, and configured to transmit the received signal converted to the digital signal to the receiving memory 34.

The body portion 30 is also provided with the receiving memory 34. The receiving memory 34 is configured to temporary store the received signal converted to the digital signal. The receiving memory 34 is connected with a data processing portion 35, and configured to transmit a stored acoustic signal to the data processing portion 35.

The body portion 30 is also provided with the data processing portion 35. The data processing portion 35 is connected with an acoustic image reconstruction portion 51, and configured to transmit data of the acoustic wave A1 to the acoustic image reconstruction portion 51. The data processing portion 35 is also connected with an ultrasonic image reconstruction portion 54, and configured to transmit data of the ultrasonic wave B2 to the ultrasonic image reconstruction portion 54.

The body portion 30 is also provided with the acoustic image reconstruction portion 51. The acoustic image reconstruction portion 51 is configured to perform processing of reconstructing the acquired data of the acoustic wave A1 as an image.

The acoustic image reconstruction portion 51 is connected with a wave detection/logarithmic converter 52, and configured to transmit the data of the acoustic wave A1 reconstructed as the image to the wave detection/logarithmic converter 52.

The body portion 30 is also provided with the wave detection/logarithmic converter 52. The wave detection/logarithmic converter 52 is configured to perform waveform processing of the data reconstructed as the image. Further, the wave detection/logarithmic converter 52 is connected with an acoustic image construction portion 53, and configured to transmit the data subjected to the waveform processing thereto.

The body portion 30 is also provided with the acoustic image construction portion 53. The acoustic image construction portion 53 is configured to perform processing of constructing a tomographic image in the specimen 10 on the basis of the data subjected to the waveform processing. Further, the acoustic image construction portion 53 is connected with an image synthesis portion 57, and configured to transmit the tomographic image based on the acoustic wave A1 thereto.

The body portion 30 is also provided with the ultrasonic image reconstruction portion 54, a wave detection/logarithmic converter 55, an ultrasonic image construction portion 56 and the image synthesis portion 57. The ultrasonic image reconstruction portion 54 is configured to perform processing of reconstructing the data of the ultrasonic wave B2 acquired from the data processing portion 35 as an image. Further, the ultrasonic image reconstruction portion 54 is configured to transmit a tomographic image based on the ultrasonic wave B2 to the image synthesis portion 57 through the wave detection/logarithmic converter 55 and the ultrasonic image construction portion 56.

The image synthesis portion 57 is configured to perform processing of synthesizing the tomographic images based on the acoustic wave A1 and the ultrasonic wave B2 and to output a synthesized image to the image display portion 40.

The body portion 30 is also provided with an operation portion 58. The operation portion 58 is configured to be capable of accepting operations of the operator. For example, the operation portion 58 is configured to acquire operation information related to on-off operations of a power source for the photoacoustic imager 100, start and end of observation (application of light and the ultrasonic wave B1) and the like and to transmit the acquired operation information to the control portion 31.

The image display portion 40 is constituted of a liquid crystal panel or the like, and configured to display images acquired from the body portion 30.

Details of operations of the light source driving portion 22 (the control portion 31) in the photoacoustic imager 100 according to the first embodiment are now described with reference to FIG. 3.

(Normal Time)

First, operations at a normal time of the light-emitting diode elements 21a are described. In the photoacoustic imager 100, the DC-DC converter 22a applies raised voltage (the driving voltage V_{D1}, about 250 V) to the anode sides of the light-emitting diode elements 21a at a time t1.

Then, the control portion 31 transmits a pulse control signal (light trigger signal) for turning on the switch portion SW at a time t2. Thus, current flows in the light-emitting diode elements 21a (the light-emitting diode element groups 21b), whereby the light-emitting diode elements 21a emit light. In a period (ON-period t) when the switch portion SW is in an ON-state, current flows in the light-emitting diode elements 21a, whereby voltage (potential) on the cathode sides of the light-emitting diode elements 21a is reduced. In practice, the voltage on the cathode sides of the light-emitting diode elements 21a does not reach 0 V (voltage V_{C}) due to on-resistance of the switch portion SW. Thus, the forward voltage value V_{F} (voltage between anodes and cathodes) detected by the forward voltage detection portion 23 becomes V_{F1} (V_{D1}—V_{C}).

Then, the control portion 31 transmits a pulse control signal (light trigger signal) for turning off the switch portion SW at a time t3. Thus, no current flows in the light-emitting diode elements 21a (the light-emitting diode element groups 21b), and the light-emitting diode elements 21a stop emitting light. In other words, ON- and OFF-states of the light-emitting diode elements 21a are controlled on the basis of the light trigger signal. In the period when the switch portion SW is in an OFF-state, no current flows in the light-emitting diode elements 21a, and hence the voltage (potential) on the cathode sides of the light-emitting diode elements 21a theoretically equals the voltage (V_{D1}) on the anode sides. According to the first embodiment, the switch portion SW is constituted of the FET, and hence small current (leakage current of 1 mA, for example) flows also when the switch portion SW is in an OFF-state. Therefore, the voltage on the cathode sides of the light-emitting diode elements 21a is reduced by a forward voltage value V_{F01} (see FIG. 4) corresponding to the small current, and reaches V_{D3} (=V_{D1}−V_{F01}×n). V_{F01} represents the forward voltage value of one light-emitting diode element 21a in the case where the switch portion SW is in an OFF-state (in the case where the small current flows). Further, n represents the number of the serially connected light-emitting diode elements 21a. Thus, a forward voltage value V_{F2} (=V_{D1}−V_{D3}=V_{F01}×n) does not become zero.

(Upon Short Circuiting)

Operations upon short circuiting of the light-emitting diode elements 21a are now described.

It is assumed that some of the plurality of light-emitting diode elements 21a are short-circuited at a time t4 when the switch portion SW is in an ON-state. In this case, the forward voltage values of the short-circuited light-emitting diode elements 21a substantially become zero, and hence the forward voltage value V_{F} detected by the forward voltage detection portion 23 is reduced from the forward voltage value V_{F1} at the aforementioned normal time (when the switch portion SW is in an ON-state) to V_{F3} (=V_{F1}−V_{F02}×m) by the number (m) of the short-circuited light-emitting diode elements 21a. Further, cathode voltage is increased in response thereto. V_{F02} (see FIG. 4) represents the forward voltage value of one light-emitting diode element 21a in the case where the switch portion SW is in an ON-state (in a case where current of 15 A flows, for example). According to the first embodiment, the light source driving portion 22 stops power supply to the light-emitting diode elements 21a on the basis of that the forward voltage detection portion 23 has detected the forward voltage value V_{F} of the plurality of light-emitting diode elements 21a corresponding to short circuiting of the light-emitting diode elements 21a. More specifically, the light source driving portion 22 stops power supply to the light-emitting diode elements 21a when the forward voltage value V_{F} of the plurality of light-emitting diode elements 21a detected by the forward voltage detection portion 23 is not more than a first threshold Vₜ₁. More detailedly, the light source driving portion 22 stops power supply to the light-emitting diode elements 21a in response to a signal from the control portion 31 when the control portion 31 determines that the forward voltage value V_{F} of the plurality of light-emitting diode elements 21a detected by the forward voltage detection portion 23 is not more than the first threshold Vₜ₁. The first threshold Vₜ₁ is an example of the “prescribed threshold” in the present invention.

The first threshold Vₜ₁ is previously set to a forward voltage value (=V_{F1}−V_{F02}×4) in a case (m=4) where the number of short-circuited light-emitting diode elements 21a is at least four, for example. In other words, the light source driving portion 22 does not stop power supply to the light-emitting diode elements 21a when the number of short-circuited light-emitting diode elements 21a is less than three. The light source driving portion 22 may stop power supply to the light-emitting diode elements 21a by stopping voltage application from the DC-DC converter 22a to the light-emitting diode elements 21a or by turning off the switch portion SW.

It is assumed that some of the plurality of light-emitting diode elements 21a are short-circuited at a time t5 when the switch portion SW is in an OFF-state. In this case, the forward voltage value V_{F} detected by the forward voltage detection portion 23 is reduced from the forward voltage value V_{F2} at the aforementioned normal time (when the switch portion SW is in an OFF-state) by the number (m) of the short-circuited light-emitting diode elements 21a, to become V_{F4} (=V_{F2}−V_{F01}×m). According to the first embodiment, the light source driving portion 22 stops power supply to the light-emitting diode elements 21a when the forward voltage value V_{F} of the plurality of light-emitting diode elements 21a detected by the forward voltage detection portion 23 is not more than a second threshold Vₜ₂. The second threshold Vₜ₂ is previously set to a forward voltage value (=V_{F2}−V_{F01}×4) in a case (m=4) where the number of short-circuited light-emitting diode elements 21a is at least four, for example. The second threshold Vₜ₂ is an example of the “prescribed threshold” in the present invention.

In other words, the light source driving portion 22 is configured to stop power supply to the light-emitting diode elements 21a on the basis of that the forward voltage detection portion 23 has detected the forward voltage value V_{F} of the plurality of light-emitting diode elements 21a corresponding to short circuiting of the light-emitting diode elements 21a in both of an ON-period when the light-emitting diode elements 21a emit light and an OFF-period when the light-emitting diode elements 21a emit no light according to the first embodiment.

According to the first embodiment, the following effects can be attained:

According to the first embodiment, as hereinabove described, the light source driving portion 22 is configured to stop power supply to the light-emitting diode elements 21a on the basis of that the forward voltage detection portion 23 has detected the forward voltage value V_{F} of the plurality of light-emitting diode elements 21a corresponding to short circuiting of the light-emitting diode elements 21a. When any one of the plurality of light-emitting diode elements 21a is short-circuited, the total forward voltage value V_{F} of the plurality of light-emitting diode elements 21a is reduced in response thereto. Therefore, when the light source driving portion 22 stops power supply to the light-emitting diode elements 21a on the basis of that the forward voltage detection portion 23 has detected the forward voltage value V_{F} of the plurality of light-emitting diode elements 21a corresponding to short circuiting of the light-emitting diode elements 21a as hereinabove described, the light-emitting diode elements 21a are prevented from emitting light in the state where any one of the light-emitting diode elements 21a is short-circuited, whereby the photoacoustic imager 100 can be prevented from continuously emitting light in a state where the quantities of light from the light-emitting diode elements 21a are insufficient.

According to the first embodiment, as hereinabove described, the light source driving portion 22 is configured to stop power supply to the light-emitting diode elements 21a when the forward voltage value V_{F} of the plurality of light-emitting diode elements 21a detected by the forward voltage detection portion 23 is not more than the prescribed threshold (the first threshold Vₜ₁ or the second threshold Vₜ₂). Even if one or more of the light-emitting diode elements 21a are short-circuited, the quantities of light may not be insufficient (within tolerance). The light source driving portion 22 is configured to stop power supply to the light-emitting diode elements 21a when the forward voltage value V_{F} of the plurality of light-emitting diode elements 21a detected by the forward voltage detection portion 23 is not more than the prescribed threshold (the first threshold Vₜ₁ or the second threshold Vₜ₂) as described above, whereby the same can properly stop power supply to the light-emitting diode elements 21a when the quantities of light are insufficient (out of tolerance).

According to the first embodiment, as hereinabove described, the prescribed threshold is set to the forward voltage value V_{F} in a case where at least two (according to the first embodiment, four) light-emitting diode elements 21a are short-circuited. Thus, the light source driving portion 22 can be prevented from stopping power supply to the light-emitting diode elements 21a when the number of short-circuited light-emitting diode elements 21a corresponds to that causing no insufficiency in the quantities of light.

According to the first embodiment, as hereinabove described, the light source driving portion 22 is configured to stop power supply to the light-emitting diode elements 21a on the basis of that the forward voltage detection portion 23 has detected the forward voltage value V_{F} of the plurality of light-emitting diode elements 21a corresponding to short circuiting of the light-emitting diode elements 21a at least in an OFF-period when the light-emitting diode elements 21a emit no light. Thus, the forward voltage detection portion 23 can easily detect the forward voltage value V_{F} dissimilarly to a case of detecting the forward voltage value V_{F} of the plurality of light-emitting diode elements 21a only in a relatively short ON-period when the light-emitting diode elements 21a emit light.

According to the first embodiment, as hereinabove described, the light source driving portion 22 is configured to stop power supply to the light-emitting diode elements 21a in both of a case where the forward voltage value V_{F} of the plurality of light-emitting diode elements 21a detected by the forward voltage detection portion 23 is not more than the first threshold Vₜ₁ in an ON-period when the light-emitting diode elements 21a emit light and a case where the forward voltage value V_{F} of the plurality of light-emitting diode elements 21a detected by the forward voltage detection portion 23 is not more than the second threshold Vₜ₂ in an OFF-period when the light-emitting diode elements 21a emit no light. Thus, the light source driving portion 22 stops power supply to the light-emitting diode elements 21a in both of ON- and OFF-periods in a case where the light-emitting diode elements 21a are short-circuited, whereby the light source driving portion 22 can be more prevented from continuously emitting light in a state where the quantities of light from the light-emitting diode elements 21a are insufficient.

According to the first embodiment, as hereinabove described, the photoacoustic imager 100 is provided with the switch portion SW consisting of the field-effect transistor having the first end connected to the light-emitting diode elements 21a and the grounded second end. Small current (leakage current) flows in the field-effect transistor also in an OFF state, and hence the forward voltage value V_{F} has a constant magnitude also in an OFF-period when the light-emitting diode elements 21a emit no light. In other words, the magnitude of the forward voltage value V_{F} varies with a case where the light-emitting diode elements 21a are short-circuited and a case where the same are not short-circuited also in an OFF-period when the light-emitting diode elements 21a emit no light. When the switch portion SW is constituted of the field-effect transistor, therefore, the photoacoustic imager 100 can detect short circuiting of the light-emitting diode elements 21a also in an OFF-period when the light-emitting diode elements 21a emit no light.

According to the first embodiment, as hereinabove described, the forward voltage detection portion 23 is provided between the light source driving portion 22 and the light-emitting diode elements 21a. Thus, the forward voltage detection portion 23 can easily acquire the forward voltage value V_{F} of the light-emitting diode elements 21a on the basis of the difference between the voltage of the light source driving portion 22 and that of the light-emitting diode elements 21a.

According to the first embodiment, as hereinabove described, the forward voltage detection portion 23 is configured to detect the sum of the forward voltage values V_{F} of the plurality of light-emitting diode elements 21a. The forward voltage value V_{F} per light-emitting diode element 21a is previously knowable, and hence the control portion 31 can easily calculate the number of short-circuited light-emitting diode elements 21a from the total forward voltage value V_{F} of the plurality of light-emitting diode elements 21a. In other words, the control portion 31 can calculate the number of short-circuited light-emitting diode elements 21a without detecting the individual forward voltage values V_{F} of the plurality of light-emitting diode elements 21a.

According to the first embodiment, as hereinabove described, the forward voltage detection portion 23 is configured to acquire voltage values on the basis of the sampling trigger signal synchronized with the light trigger signal controlling ON- and OFF-states of the light-emitting diode elements 21a. Thus, the forward voltage detection portion 23 can acquire the voltage values in synchronization with ON- and OFF-states of the light-emitting diode elements 21a.

According to the first embodiment, as hereinabove described, the light source portion 21 is provided with the light-emitting diode elements 21a. Thus, the range of light application is relatively hard to change even if misregistration is caused, since the light-emitting diode elements 21a are lower in directivity as compared with light-emitting semiconductor elements emitting laser beams. Further, the photoacoustic imager 100 requires neither precise alignment (registration) of optical members nor an optical bench or a strong housing for preventing characteristic fluctuation resulting from vibration of optical systems, dissimilarly to a case of employing light-emitting semiconductor elements emitting laser beams. Consequently, the photoacoustic imager 100 can be prevented from size increase and complication in structure due to the nonrequirement for precise alignment of optical members and for an optical bench or a strong housing.

### Second Embodiment

The structure of a photoacoustic imager 200 according to a second embodiment of the present invention is now described with reference to FIGS. 1 and 3. According to the second embodiment, a forward voltage detection portion 223 detects a forward voltage value V_{F} only in an OFF-period, dissimilarly to the aforementioned first embodiment in which the forward voltage detection portion 23 detects the forward voltage value V_{F} in both of ON- and OFF-periods.

As shown in FIG. 1, the photoacoustic imager 200 according to the second embodiment is provided with a light source driving portion 222 and a control portion 231.

According to the second embodiment, the light source driving portion 222 (the control portion 231) is configured to stop power supply to a plurality of light-emitting diode elements 21a on the basis of that the forward voltage detection portion 223 has detected the forward voltage value V_{F} of the plurality of light-emitting diode elements 21a only in an OFF-period when the light-emitting diode elements 21a emit no light. Thus, the light source driving portion 222 does not stop power supply to the light-emitting diode elements 21a at a time t4 (when any of the light-emitting diode elements 21a is short-circuited in a period when a switch portion SW is in an ON-state) shown in FIG. 3.

The remaining structure of the second embodiment is similar to that of the aforementioned first embodiment.

According to the second embodiment, the following effect can be attained:

According to the second embodiment, as hereinabove described, the light source driving portion 222 is configured to stop power supply to the light-emitting diode elements 21a on the basis of that the forward voltage detection portion 223 has detected the forward voltage value V_{F} of the plurality of light-emitting diode elements 21a corresponding to short circuiting of the light-emitting diode elements 21a only in an OFF-period when the light-emitting diode elements 21a emit no light. In a case of detecting the forward voltage value V_{F} of the plurality of light-emitting diode elements 21a in a relatively short ON-period when the light-emitting diode elements 21a emit light, the photoacoustic imager 200 may separately require a circuit (a sample hold circuit, for example) holding the forward voltage value V_{F} until the forward voltage detection portion 223 performs an operation of detecting the forward voltage value V_{F} in practice. The light source driving portion 223 is configured to stop power supply to the light-emitting diode elements 21a on the basis of that the forward voltage detection portion 223 has detected the forward voltage value V_{F} of the plurality of light-emitting diode elements 21a only in an OFF-period when the light-emitting diode elements 21a emit no light as described above so that the forward voltage detection portion 223 detects the forward voltage value V_{F} of the plurality of light-emitting diode elements 21a in a relatively long OFF-period, whereby the forward voltage detection portion 223 can easily detect the forward voltage value V_{F} with no requirement for a circuit for holding the forward voltage value V_{F}.

The remaining effects of the second embodiment are similar to those of the aforementioned first embodiment.

### Third Embodiment

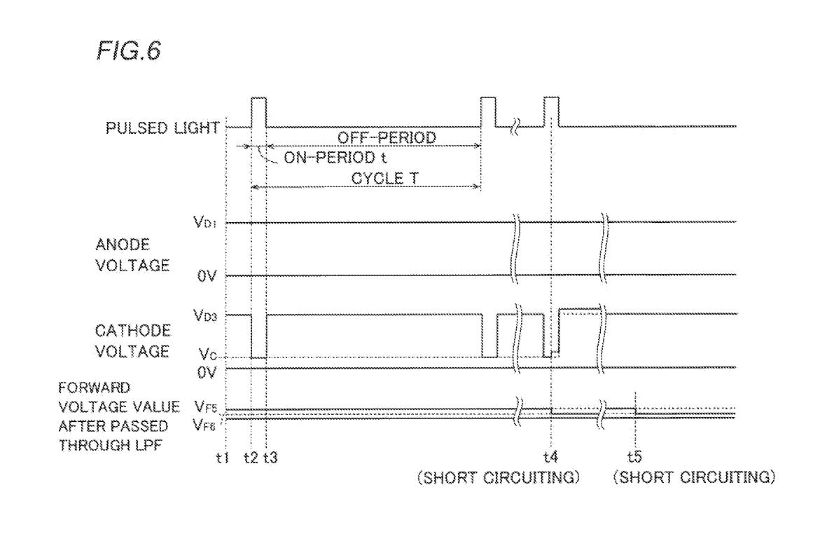

The structure of a photoacoustic imager 300 according to a third embodiment of the present invention is now described with reference to FIGS. 5 and 6. According to the third embodiment, a low-pass filter (LPF) 301 is provided between light-emitting diode elements 21a and a forward voltage detection portion 23, dissimilarly to the aforementioned first and second embodiments.

In the photoacoustic imager 300, the low-pass filter 301 is provided between the light-emitting diode elements 21a (a light source portion 21) and the forward voltage detection portion 23, as shown in FIG. 5. The photoacoustic imager 300 is so configured that a forward voltage value V_{F} of the light-emitting diode elements 21a detected by the forward voltage detection portion 23 through the low-pass filter 301 is transmitted to a control portion 31. Thus, the forward voltage value V_{F} after passed through the low-pass filter 301 is substantially constant (a forward voltage value V_{F5}) in both of an ON-period when the light-emitting diode elements 21a emit light and an OFF-period when the light-emitting diode elements 21a emit no light, as shown in FIG. 6. Further, the photoacoustic imager 300 is so configured that the forward voltage detection portion 23 detects (monitors) the forward voltage value V_{F} of the light-emitting diode elements 21a in both of an ON-period when the light-emitting diode elements 21a emit light and an OFF-period when the light-emitting diode elements 21a emit no light.

When some of the plurality of light-emitting diode elements 21a are short-circuited at a time t4 in an ON-period (or at a time t5 in an OFF-period), the forward voltage value V_{F5} at a normal time is reduced to V_{F6} by the number (m) of the short-circuited light-emitting diode elements 21a. Further, cathode voltage is increased in response thereto. A light source driving portion 22 stops power supply to the light-emitting diode elements 21a when the forward voltage value V_{F} of the plurality of light-emitting diode elements 21a detected by the forward voltage detection portion 23 is not more than a common third threshold Vₜ₃ in ON- and OFF-periods of the light-emitting diode elements 21a. The third threshold Vₜ₃ is previously set to a forward voltage value in a case (m=4) where the number of short-circuited light-emitting diode elements 21a is at least four, for example. The third threshold Vₜ₃ is an example of the “prescribed threshold” in the present invention.

The remaining structure of the third embodiment is similar to that of the aforementioned first embodiment.

According to the third embodiment, the following effects can be attained:

According to the third embodiment, as hereinabove described, the low-pass filter 301 is provided between the light-emitting diode elements 21a and the forward voltage detection portion 23, and the light source driving portion 22 is configured to stop power supply to the light-emitting diode elements 21a on the basis of that the forward voltage detection portion 23 has detected the forward voltage value V_{F} of the plurality of light-emitting diode elements 21a corresponding to short circuiting of the light-emitting diode elements 21a through the low-pass filter 301. Thus, the (relatively large) forward voltage value V_{F} in an ON-period when the light-emitting diode elements 21a emit light and the (relatively small) forward voltage value V_{F} in an OFF-period when the light-emitting diode elements 21a emit no light are rendered substantially constant through the low-pass filter 301, whereby the forward voltage detection portion 23 can easily detect the forward voltage value V_{F} regardless of an ON- or OFF-period.

According to the third embodiment, as hereinabove described, the light source driving portion 22 is configured to stop power supply to the light-emitting diode elements 21a when the forward voltage value V_{F} of the plurality of light-emitting diode elements 21a detected by the forward voltage detection portion 23 through the low-pass filter 301 is not more than the common third threshold Vₜ₃ in an ON-period when the light-emitting diode elements 21a emit light and an OFF-period when the light-emitting diode elements 21a emit no light. The forward voltage detection portion 23 detects the forward voltage value V_{F} through the low-pass filter 301 for rendering the forward voltage value V_{F} in an ON-period and that in an OFF-period substantially constant, whereby the light source driving portion 22 can properly stop power supply to the light-emitting diode elements 21a by employing the third threshold Vₜ₃.

The remaining effects of the third embodiment are similar to those of the aforementioned first embodiment.

### Fourth Embodiment

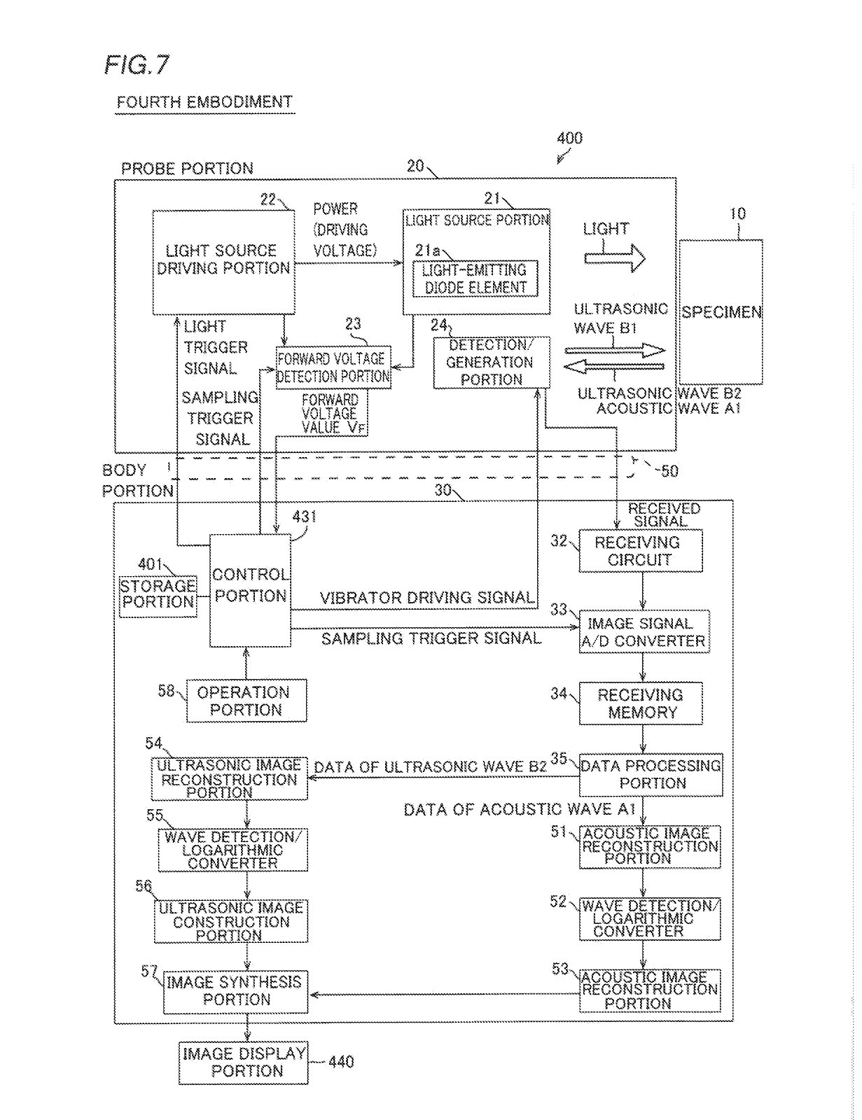

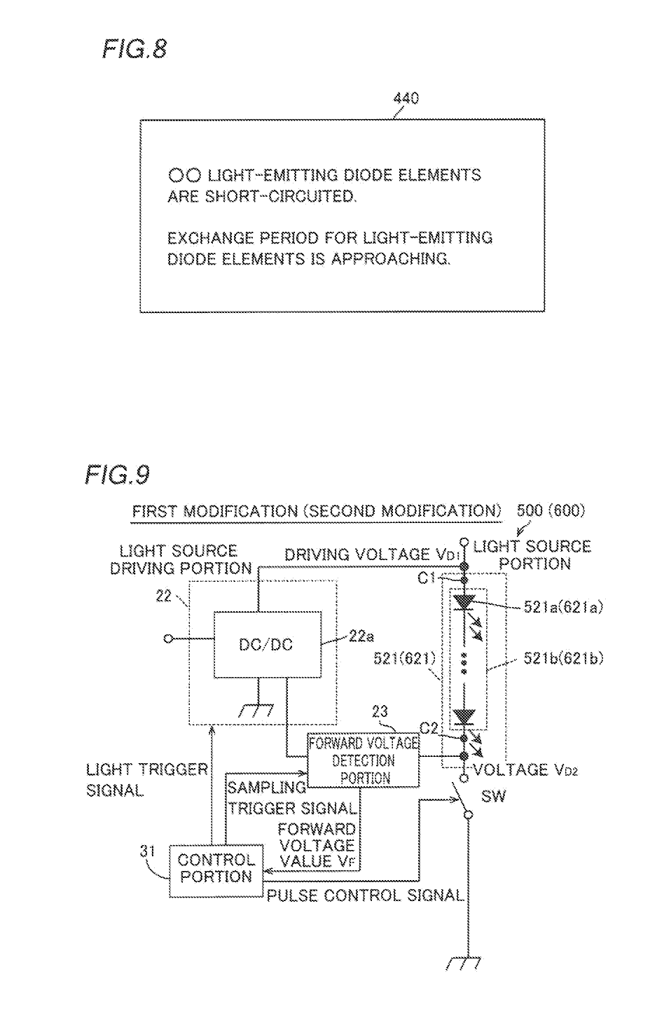

The structure of a photoacoustic imager 400 according to a fourth embodiment of the present invention is now described with reference to FIGS. 7 and 8. According to the fourth embodiment, the photoacoustic imager 400 is so configured that an image display portion 440 displays information related to a plurality of light-emitting diode elements 21a on the basis of a forward voltage value V_{F} of the plurality of light-emitting diode elements 21a, dissimilarly to the aforementioned first to third embodiments. The image display portion 440 is an example of the “display portion” in the present invention.

In the photoacoustic imager 400 according to the fourth embodiment, a forward voltage detection portion 23 is configured to input the forward voltage value V_{F} of the light-emitting diode elements 21a into a control portion 431 as the information related to the light-emitting diode elements 21a, as shown in FIG. 7. The forward voltage value V_{F} of the light-emitting diode elements 21a received from the forward voltage detection portion 23 consists of A-D-converted digital data. The control portion 431 is configured to acquire (calculate) the number of short-circuited light-emitting diode elements 21a from the digital data. More specifically, a value 1 (=(V_{F1}−V_{F})/V_{F02}) obtained by dividing difference between a forward voltage value V_{F1} (see FIG. 3) at a normal time when the light-emitting diode elements 21a are not short-circuited and the acquired forward voltage value V_{F} by a forward voltage value _{VF02} (see FIG. 4) per light-emitting diode element 21a corresponds to the number of short-circuited light-emitting diode elements 21a at a time when the light-emitting diode elements 21a are in ON-states. At a time when light-emitting diode elements 21a are in OFF-states, a value 1 (=(V_{F2} −V_{F})/V_{F01}) obtained by dividing difference between a forward voltage value V_{F2} (see FIG. 3) at a normal time when the light-emitting diode elements 21a are not short-circuited and the acquired forward voltage value V_{F} by a forward voltage value _{VF01} (see FIG. 4) per light-emitting diode element 21a corresponds to the number of short-circuited light-emitting diode elements 21a.

According to the fourth embodiment, the image display portion 440 is configured to display states of the light-emitting diode elements 21a on the basis of that the forward voltage detection portion 23 has detected the forward voltage value V_{F} of the plurality of light-emitting diode elements 21a corresponding to short circuiting of the light-emitting diode elements 21a, as shown in FIG. 8. More specifically, the image display portion 440 is configured to display the number 1 (◯◯) of the short-circuited light-emitting diode element 21a calculated by the control portion 31.

Further, the photoacoustic imager 400 is provided with a storage portion 401, as shown in FIG. 7. The photoacoustic imager 400 is so configured that the storage portion 401 stores the information (the forward voltage value V_{F} of the light-emitting diode elements 21a) related to the light-emitting diode elements 21a transmitted to the control portion 431. The control portion 431 is configured to calculate the lifetime of the light-emitting diode elements 21, an exchange period therefor and the like from the hysteresis of the information related to the light-emitting diode elements 21a stored in the storage portion 401 and to display the same on the image display portion 440. For example, the control portion 431 is configured to display that the period for exchanging the light-emitting diode elements 21a (the lifetime of the light-emitting diode elements 21a) is approaching as shown in FIG. 8, in a case where a period when the number of short-circuited light-emitting diode elements 21a exceeds a tolerance approaches.

The image display portion 440 may display the states of the light-emitting diode elements 21a when the photoacoustic imager 400 is driven (a state where emission from the light-emitting diode elements 21a and stoppage of the emission are repeated) or power is applied to the photoacoustic imager 400 (initialization).

The remaining structure of the fourth embodiment is similar to that of the aforementioned first embodiment.

According to the fourth embodiment, the following effects can be attained:

According to the fourth embodiment, as hereinabove described, the photoacoustic imager 400 is provided with the image display portion 440 displaying the states of the light-emitting diode elements 21a on the basis of that the forward voltage detection portion 23 has detected the forward voltage value V_{F} of the plurality of light-emitting diode elements 21a corresponding to short circuiting of the light-emitting diode elements 21a. Thus, the photoacoustic imager 400 can easily make the user visually recognize the states of the light-emitting diode elements 21a (such as the number of short-circuited light-emitting diode elements 21a based on the forward voltage value V_{F}, for example). Consequently, the photoacoustic imager 400 can prompt the user to exchange the light-emitting diode elements 21a.

According to the fourth embodiment, as hereinabove described, the photoacoustic imager 400 is provided with the control portion 431 calculating the number of short-circuited light-emitting diode elements 21a on the basis of the forward voltage value V_{F} of the plurality of light-emitting diode elements 21a detected by the forward voltage detection portion 23 and the forward voltage value (V_{F01} or V_{F02}) per light-emitting diode element 21a, and the image display portion 440 is configured to display the number of the short-circuited light-emitting diode elements 21a calculated by the control portion 431. Thus, the photoacoustic imager 400 can make the user recognize that the exchange period for the light-emitting diode elements 21a is approaching.

According to the fourth embodiment, as hereinabove described, the photoacoustic imager 400 is provided with the storage portion 401 storing the states of the light-emitting diode elements 21a and the control portion 431 calculating the exchange period for the light-emitting diode elements 21a on the basis of the states of the light-emitting diode elements 21a stored in the storage portion 401, and the image display portion 440 is configured to display the exchange period for the light-emitting diode elements 21a calculated by the control portion 431. Thus, the photoacoustic imager 400 can make the user prepare new light-emitting diode elements 21a for exchange.

According to the fourth embodiment, as hereinabove described, the image display portion 440 is configured to display the states of the light-emitting diode elements 21a when the photoacoustic imager 400 is driven or power is applied to the photoacoustic imager 400. Thus, the photoacoustic imager 400 can easily make the user recognize the states of the light-emitting diode elements 21a.

The remaining effects of the fourth embodiment are similar to those of the aforementioned first embodiment.

Although the present invention has been described and illustrated in detail, it is clearly understood that the same is by way of illustration and example only and is not to be taken by way of limitation, the spirit and scope of the present invention being limited only by the terms of the appended claims.

For example, while the photoacoustic imager employs the light-emitting diode elements as the light-emitting semiconductor elements according to the present invention in each of the aforementioned first to fourth embodiments, the present invention is not restricted to this. According to the present invention, the photoacoustic imager may alternatively be configured to employ light-emitting semiconductor elements other than the light-emitting diode elements. For example, a plurality of semiconductor laser elements 521a may be employed as the light-emitting semiconductor elements according to the present invention, as in a photoacoustic imager 500 according to a first modification shown in FIG. 9. In the photoacoustic imager 500, a light source portion 521 is provided with the plurality of serially connected semiconductor laser elements 521a, which in turn constitute semiconductor laser element groups 521b. When employing the semiconductor laser elements 521a, the photoacoustic imager 500 can apply laser beams relatively higher in directivity as compared with light-emitting diode elements to a specimen 10, whereby the same can reliably apply most part of light from the semiconductor laser elements 521a to the specimen 10. The semiconductor laser elements 521a are examples of the “light-emitting semiconductor elements” in the present invention.

Further alternatively, a plurality of organic light-emitting diode elements 621a may be employed as the light-emitting semiconductor elements according to the present invention, as in a photoacoustic imager 600 according to a second modification shown in FIG. 9. In the photoacoustic imager 600, a light source portion 621 is provided with the plurality of serially connected organic light-emitting diode elements 621a, which in turn constitute organic light-emitting diode element groups 621b. When employing the organic light-emitting diode elements 621a reducible in thickness, the light source portion 621 provided with the organic light-emitting diode elements 621a can be easily miniaturized. The organic light-emitting diode elements 621a are examples of the “light-emitting semiconductor elements” in the present invention.

While the prescribed threshold is so set that the light source driving portion stops power supply to the light-emitting diode elements when at least four light-emitting diode elements are short-circuited in each of the aforementioned first to fourth embodiments, the present invention is not restricted to this. According to the present invention, the prescribed threshold may alternatively be so set that the light source driving portion stops power supply to the light-emitting diode elements when light-emitting diode elements of at least a number other than four are short-circuited.

While the photoacoustic imager outputs information (short circuiting or the like) related to the light-emitting diode elements to the image display portion displaying tomographic images in each of the aforementioned first to fourth embodiments, the present invention is not restricted to this. According to the present invention, the photoacoustic imager may alternatively be provided with another display portion (a monitor, an indicator or the like) separately from the image display portion displaying tomographic images, for displaying the information (short circuiting or the like) related to the light-emitting diode elements, for example.

While the photoacoustic imager employs the switch portion consisting of the FET in each of the aforementioned first to fourth embodiments, the present invention is not restricted to this. According to the present invention, the photoacoustic imager may alternatively employ a bipolar transistor or an IGBT (insulated gate bipolar transistor) as the switch portion, for example.

What is claimed is:

1. A photoacoustic imager comprising:
a detection/generation portion capable of detecting an acoustic wave generated due to absorption of light by a detection object in a specimen and generating an ultrasonic wave to be applied to the specimen;
a plurality of light-emitting semiconductor elements provided in the vicinity of the detection/generation portion and capable of applying light to the specimen;
a light source driving portion supplying power for making the light-emitting semiconductor elements generate light to the light-emitting semiconductor elements; and
a forward voltage detection portion capable of detecting forward voltage values of the light-emitting semiconductor elements, wherein
the light source driving portion is configured to stop power supply to the light-emitting semiconductor elements on the basis of that the forward voltage detection portion has detected forward voltage values of the plurality of light-emitting semiconductor elements corresponding to short circuiting of the light-emitting semiconductor elements.

2. The photoacoustic imager according to claim 1, wherein
the light source driving portion is configured to stop power supply to the light-emitting semiconductor elements in a case where the forward voltage values of the plurality of light-emitting semiconductor elements detected by the forward voltage detection portion are not more than a prescribed threshold.

3. The photoacoustic imager according to claim 2, wherein
the prescribed threshold is set to a forward voltage value in a case where at least two light-emitting semiconductor elements are short-circuited.

4. The photoacoustic imager according to claim 1, wherein
the light source driving portion is configured to stop power supply to the light-emitting semiconductor elements on the basis of that the forward voltage detection portion has detected the forward voltage values of the plurality of light-emitting semiconductor elements corresponding to short circuiting of the light-emitting semiconductor elements at least in an OFF-period when the light-emitting semiconductor elements emit no light.

5. The photoacoustic imager according to claim 4, wherein
the light source driving portion is configured to stop power supply to the light-emitting semiconductor elements in both of a case where the forward voltage values of the plurality of light-emitting semiconductor elements detected by the forward voltage detection portion are not more than a first threshold in an ON-period when the light-emitting semiconductor elements emit light and a case where the forward voltage values of the plurality of light-emitting semiconductor elements detected by the forward voltage detection portion are not more than a second threshold in an OFF-period when the light-emitting semiconductor elements emit no light.

6. The photoacoustic imager according to claim 4, wherein
the light source driving portion is configured to stop power supply to the light-emitting semiconductor elements in a case where the forward voltage values of the plurality of light-emitting semiconductor elements detected by the forward voltage detection portion are not more than a second threshold only in an OFF-period when the light-emitting semiconductor elements emit no light.

7. The photoacoustic imager according to claim 1, further comprising a low-pass filter provided between the light-emitting semiconductor elements and the forward voltage detection portion, wherein
the light source driving portion is configured to stop power supply to the light-emitting semiconductor elements on the basis of that the forward voltage detection portion has detected the forward voltage values of the plurality of light-emitting semiconductor elements corresponding to short circuiting of the light-emitting semiconductor elements through the low-pass filter.

8. The photoacoustic imager according to claim 7, wherein
the light source driving portion is configured to stop power supply to the light-emitting semiconductor elements in a case where the forward voltage values of the plurality of light-emitting semiconductor elements detected by the forward voltage detection portion through the low-pass filter are not more than a common third threshold in an ON-period when the light-emitting semiconductor elements emit light and in an OFF-period when the light-emitting semiconductor elements emit no light.

9. The photoacoustic imager according to claim 1, further comprising a display portion displaying states of the light-emitting semiconductor elements on the basis of that the forward voltage detection portion has detected the forward voltage values of the plurality of light-emitting semiconductor elements corresponding to short circuiting of the light-emitting semiconductor elements.

10. The photoacoustic imager according to claim 9, further comprising a control portion calculating the number of short-circuited light-emitting semiconductor elements on the basis of the forward voltage values of the plurality of light-emitting semiconductor elements detected by the forward voltage detection portion and a forward voltage value per light-emitting semiconductor element, wherein
the display portion is configured to display the number of the short-circuited light-emitting semiconductor elements calculated by the control portion.

11. The photoacoustic imager according to claim 9, further comprising:
a storage portion storing states of the light-emitting semiconductor elements; and
a control portion calculating an exchange period for the light-emitting semiconductor elements on the basis of the states of the light-emitting semiconductor elements stored in the storage portion, wherein
the display portion is configured to display the exchange period for the light-emitting semiconductor elements calculated by the control portion.

12. The photoacoustic imager according to claim 9, wherein
the display portion is configured to display the states of the light-emitting semiconductor elements when the photoacoustic imager is driven or power is applied to the photoacoustic imager.

13. The photoacoustic imager according to claim 1, further comprising a switch portion having a first end connected to the light-emitting semiconductor elements and a grounded second end, wherein
the switch portion consists of a field-effect transistor.

14. The photoacoustic imager according to claim 1, wherein
the forward voltage detection portion is provided between the light source driving portion and the light-emitting semiconductor elements.

15. The photoacoustic imager according to claim 1, wherein
the forward voltage detection portion is configured to detect the sum of the forward voltage values of the plurality of light-emitting semiconductor elements.

16. The photoacoustic imager according to claim 1, wherein
the forward voltage detection portion is configured to acquire voltage values on the basis of a sampling trigger signal synchronized with a light trigger signal controlling ON- and OFF-states of the light-emitting semiconductor elements.

17. The photoacoustic imager according to claim 1, wherein
the plurality of light-emitting semiconductor elements are connected in series with each other.

18. The photoacoustic imager according to claim 1, wherein
the light-emitting semiconductor elements are constituted of light-emitting diode elements.

19. The photoacoustic imager according to claim 1, wherein
the light-emitting semiconductor elements are constituted of semiconductor laser elements.

20. The photoacoustic imager according to claim 1, wherein
the light-emitting semiconductor elements are constituted of organic light-emitting diode elements.
